(19) United States
(12) Patent Application Publication
(10) Pub. No.: US 2005/0180968 A1
(43) Pub. Date: Aug. 18, 2005

(54) Use of relaxin for stimulating the development of activated human T cells into TH1-like effectors

(76) Inventor: Mario Bagazzi, Firenze (IT)

Correspondence Address: McGLEW AND TUTTLE;John James McGlew, Scarborough Station, Scarborough, NY, 10510-9227, US

(21) Appl. No.: 11/108,008

(22) Filed: Apr. 15, 2005

Related U.S. Application Data
(63) Continuation of application No. 09/606,569, filed on Jun. 29, 2000, now abandoned.

(51) Int. Cl. A61K 38/17; A61K 39/395

(52) U.S. Cl. 424/130.1; 514/2

(57) ABSTRACT

Relaxin (RLX) is shown to be effective in stimulating the development of activated human T cells into Th1-like effectors, for counteracting Th2-dominated disorders and for regulating immune homeostasis during pregnancy. Methods are contemplated of administering RLX or a derivative thereof to a human patient to treat a Th2-dominated disease, such as by enhancing Th1 response of the immunological system, or by inducing endogenous IFN-γ production; to inhibit a pathogenic Th2 response by inducing endogenous IFN-γ production; and to stimulate development of activated human T cells into Th1-like effectors. In the case of a pregnant female patient, a method is contemplated of administering RLX or a derivative thereof to regulate immune homeostasis.

### CROSS REFERENCE TO RELATED APPLICATIONS

This application is a continuation of application Ser. No. 09/606,569 filed Jun. 29, 2000, the entire contents of which are incorporated herein by reference.

### TECHNICAL FIELD

The present invention relates to a novel use of relaxin (hereinafter indicated also shortly as RLX), a 6-kDa polypeptide hormone predominantly produced by the corpus luteum during pregnancy, as well as by ovarian follicle and decidua, which is mainly active on the female reproductive system.

Recently, it has been shown that RLX exhibits additional, multiple effects on organs other than the reproductive ones. In particular, RLX is able to modulate the biological activity of bone marrow-derived cells, such as mast cells, platelets and granulocytes, and to reduce the allergic asthma-like reaction elicited by antigen inhalation. The use of RLX for the treatment of circulatory vascular ischemic diseases has also been suggested. These uses of RLX are disclosed and discussed in U.S. Pat. No. 5,952,296.

The present invention relates to different and unexpected medical uses of RLX.

### BACKGROUND

CD4⁺ Th lymphocytes can be classified into different functional subsets based on their profile of cytokine production. Th1 cells produce IFN-?, IL-2 and TNF-?, and promote the production of opsonizing and complement-fixing antibodies, macrophage activation, antibody-dependent cellular cytotoxicity and DTH. On the otherside, Th2 cells produce IL-4, IL-5, IL-6, IL-9, IL-10 and IL-13 and provide optimal help for humoral immune responses, including IgE isotype switching, and mucosal immunity, through mast cell and eosinophil differentiation and facilitation of IgA synthesis. In addition, some Th2-derived cytokines, such as IL-4, IL-10 and IL-13, inhibit several macrophage functions. The development of Th1- or Th2-dominated responses depends on several factors, the most critical being cytokines produced in the microenvironment during antigen presentation. IFN-?, IFN-? and IL-12 promote the differentiation of naive Th cells into the Th1 pathway, whereas IL-4 appears to be the most dominant factor for determining the Th2 polarization.

In the absence of clearly polarizing signals, CD4⁺ T cell subsets with a less differentiated lymphokine profile than Th1 or Th2 cells, designated Th0, usually arise and show intermediate effects depending upon the ratio of cytokines produced and the nature of the responding cells.

The differentiation of Th cells into polarized Th1 or Th2 cells can also be influenced by some hormones. It has been reported that the Th2 cell development and/or activity is up-regulated by glucocorticoids and progesterone, whereas Th1 cell activity is potentiated by dehydroepiandrosterone and inhibited by 25-(OH) vitamin D3.

### SUMMARY OF THE INVENTION

It has now surprisingly been observed that RLX has an effect on the differentiation of antigen-specific T cells into IFN-?- and/or IL-4-producing cells, as well as on the production of IFN-? and IL-4 induced by TCR stimulation of established T cell clones. Experiments have shown that RLX, when added to cultures of PBMC, favors the development of antigen-specific CD4⁺ T cells into T cells showing enhanced ability to produce IFN-?, without exerting any effect on the production of IL-4. In addition, RLX also increases both IFN-? mRNA expression and IFN-? production induced byTCR stimulation of established CD4⁺ T cell clones, suggesting that this hormone can directly influence both the differentiation and function of CD4⁺ effector T lymphocytes.

In essence, relaxin or derivatives thereof can be used as pharmaceutically active compounds in medicaments useful for the treatment of Th2-dominated disorders and for the regulation of the immune homeostasis during pregnancy.

The promoting effect of RLX on the development of IFN-?-producing cells was not due to relaxin-induced release of IL-12 and/or IFN-? by antigen-presenting cells, as proven by the results of the tests discussed hereinafter.

The effect of RLX on the differentiation of antigen-specific T cells into IFN-?- and/or IL4-producing cells, has been analyzed, as well as on the production of IFN-? and IL-4 induced by TCR stimulation of established T cell clones. The results show that RLX, when added to cultures of PBMC, favors the development of antigen-specific CD4⁺ T cells into T cells showing enhanced ability to produce IFN-?, without exerting any effect on the production of IL-4. In addition, RLX also increases both IFN-? mRNA expression and IFN-? production induced by TCR stimulation of established CD4⁺ T cell clones, suggesting that this hormone can directly influence both the differentiation and function of CD4⁺ effector T lymphocytes.

### BRIEF DESCRIPTION OF THE DRAWINGS

The attached drawings show the results of the experiments on the novel use of relaxin according to the present invention. More particularly:

FIG. 1 is a diagram showing the effect of RLX concentration on the production of IL-4 and IFN-? cytokines respectively;

FIG. 2 is a diagram showing the effect of RLX on IFN-? production on established CD4⁺ T cell clones;

FIG. 3 shows the result of stimulation of the CD4⁺ T cell clones with immobilized anti-CD3 antibody in the absence and in the presence of RLX;

FIGS. 4A, 4B show competitive PCR for IFN-? of a CD4⁺ T cell clone in the presence of RLX (FIG. 4A) and relevant image analysis (FIG. 4B);

FIGS. 5A and 5B show southern blot analysis result for IFN-? of CD4+T cell clones in the presence of RLX, FIG. 5B being the image analysis of the cell clones of FIG. 5A; and

FIG. 6 showing the effect of RLX.

### DETAIL DESCRIPTION

Generation of TT-Specific Short-Term T Cell Lines

TT-specific short-term T cell lines were generated from PBMC of TT-vaccinated normal donors according to a technique described in M. P. Piccinni et al <<Progesterone favors the development of human T helper cells producing Th2-type cytokines and promotes both IL-4 production and membrane CD30 expression in established TH1 cell clones>>, in J. Immmunol., 1995, 155: 128-133.

Briefly, 10⁵ PBMC in 2 ml RPMI 1640 medium supplemented with 2 mM L-glutamine, 2×10⁻⁵M 2-ME and 5% human serum (complete medium) were stimulated in 24-well flat-bottom plates for 5 days with the antigen (0.2?g/ml TT) in the absence or in the presence of RLX at concentrations of 10⁻¹⁰, 10⁻⁹, 10⁻⁸, 10⁻⁷ and 10⁻⁶ M. Human IL-2 (20 U/ml) was then added and cultures continued for another 9 days. Viable T blasts were resuspended in complete medium and tested for their antigen specificity. To this end, 2×10⁴ T blasts were seeded in microplates and cocultured for 48 h with irradiated (6000 rad) autologous PBMC (5×10⁴) in the presence of medium alone or 0.1 ? g/ml TT, respectively. After a 16-h pulse with 0.5 ?Ci [³H]thimidine, cultures where harvested and radioactivity measured by liquid scintillation. The phenotype distribution of TT-specific T cells was assessed by flow cytometer analysis.

Generation of T Cell Clones

To generate T cell clones, PBMC from normal subjects were seeded under limiting dilution conditions (0.3 cell/well) in six round-bottom microwell plates containing 105 irradiated (6000 rad) allogeneic PBMC (as feeder cells) and PHA (1% vol/vol) in a final volume of 0.2 ml complete medium supplemented with 20 U/ml IL-2 and 10% FCS. Growing microcultures where then supplemented, at weekly intervals, with 20 U/ml IL-2 and 10⁵ irradiated feeder cells. The phenotype distribution of T cell clones was assessed by flow cytometer-analysis.

Quantitation of IFN-? and IL-4 Production in Supernatants from TT-Specific Short-Term Lines and Established T Cell Clones

To quantitate cytokine production in supernatants of TT-specific shortterm T cell lines and established T cell clones, 108 T blasts were cultured in the presence of 20 ng/ml PMA plus 100 ng/ml anti-CD3 mAb. After 36 h culture, cell-free supernatants were collected, filtered, and stored in aliquots at −70° C. until used. Determination of IFN-? was performed by a commercial ELISA (Biosource International, Camarill, Calif.). IL-4 was quantified by in-house-made capture ELISA using anti IL-4 mAb bound to macrowell plates and blotinylated anti IL-4 mAb as revealing antibodies, respectively. Values of the cytokine content 5 SD over those of control supernatants obtained by stimulation of irradiated feeder cells alone were regarded as positive.

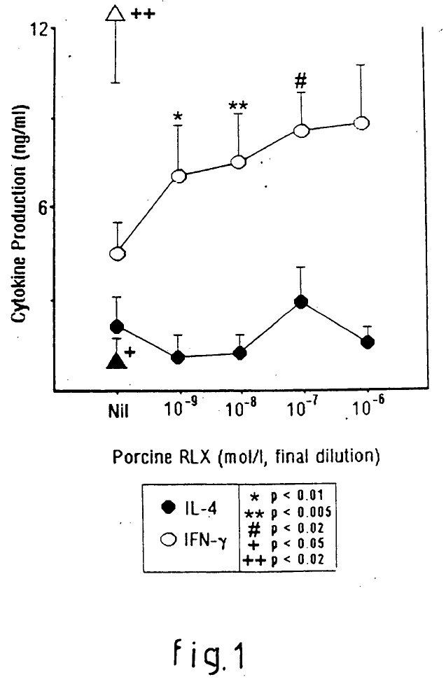

FIG. 1 shows the mean values (+SEM) of IFN-? and IL-4 produced by TT-specific T cell cultures obtained in the presence of antigen plus 200 U/ml IL-12 as a function of RLX concentration. RLX increases the production of IFN-? in TT-specific T cell lines as can be seen by the upper curve reported in FIG. 1, which increases with the concentration of RLX. On the other hand, no effect of RLX dilution can be observed on the production of IL-4.

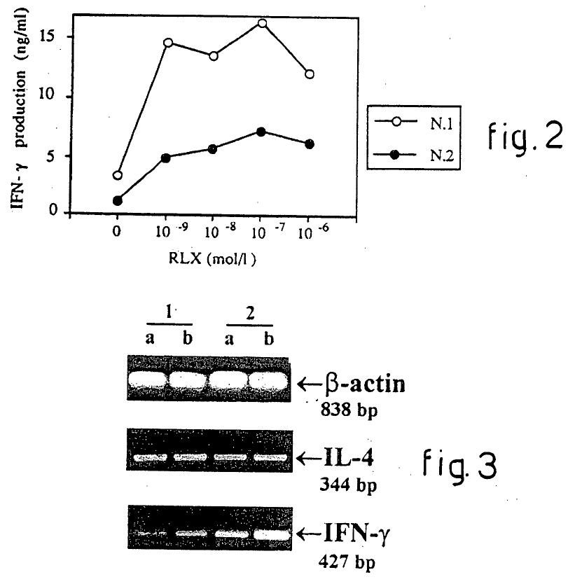

FIG. 2 shows the increase of IFN-? production in established CD4⁺ T cell clones induced by RLX. The two curves shown in FIG. 2 relate to two CD4⁺ T cell clones stimulated with immobilised anti-CD3 antibody in the presence or in the absence of different concentrations of RLX for 36 h. FIG. 3 shows the same two clones which were stimulated with immobilized anti-CD3 antibody in the absence (a) or in the presence (b) of 10′ M RLX for 12 h. Total RNA was extracted as described herein after. 1?g of RNA was reversed transcribed and cDNA were amplified and then subjected to PCR amplification with primers for IFN-? and IL4 as discussed more in detail below.

RNA Extraction and RT-PCR

To induce cytokine mRNA expression in established T cell clones for RNA extraction 2×10⁵ T blasts from each clone were cultured with insolubilized anti-CD3 (1?g/ml). After 12 h, pellets were washed four times with PBS at pH 7.2 and kept frozen until used. Total RNA was extracted using Ultraspec RNA Isolation kit (Biotex Laboratories, Inc. Houston, Tex., USA). All samples clearly showed 18S and 28S bands in a 0.8% agarose gel, indicating integrity of RNA. Following extraction, 1 ?g of RNA was reverse transcribed by M-MLV reverse transcriptase (Gibco-BRL, Gaithersburg, Md., USA). All cDNA were amplified with primers for ?-actin to test the quality of cDNA and then subjected to PCR amplification with primers for IFN-? and IL-4. In each PCR reaction a positive control and a negative control (without cDNA as template to exclude contamination) were included. PCR analysis was carried out in a DNA thermal cycler (Idaho Technology, Idaho Falls, ID, USA). Each sample was subjected to 30 cycles of amplification using 10 pM of each primer and 0.5 U ampliTaq DNA polymerase (Perkin-Elmer, Norwalk, Conn., USA) in 10?l volume in capillary glass. The cycling conditions for?-actin, IL-4 and IFN-? amplifications were 94° C. for 10 s, 66° C. for 20 s and 72° C. for 30 s with 30 cycles. Before the first cycle, samples were denatured at 94° C. for 20 s and, after the last cycle, samples were subjected to a final elongation step (72° C. for 50 s). PCR products were electrophoresed on 1.5% agarose gels and visualized by ethidium bromide staining. The size of the amplified products was evaluated by comparison with molecular weight markers run in parallel lanes.

Competitive PCR

Competitive PCRfor IFN-?was performed using a competitorcontrol fragment (PCR MIMIC; Clontech Laboratories Inc. Palo Alto, Calif., USA) according to the manufacturer's instructions. PCR MIMIC was used together with sample cDNA in the reaction mixture; sample and control cDNA were amplified with the same primers, so they competed for the same primers in the same reaction, but were distinguished on gel electrophoresis by differences in length. By knowing the amount of PCR MIMIC added to the reaction, the amount of target cDNA and therefore the initial mRNA levels could be determined. Each sample (and competitor fragment) was subjected to 25 cycles of amplification as described above.

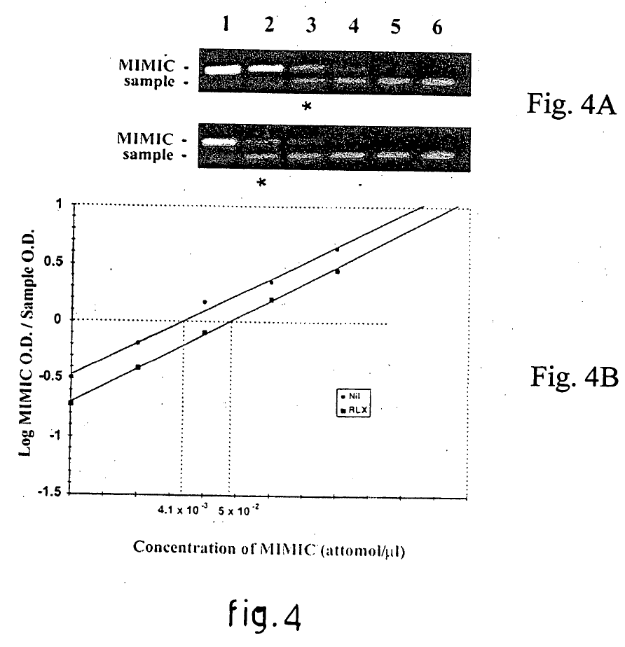

The results are shown in FIGS. 4A and 4B. More particularly, FIG. 4A shows competitive PCR for IFN-? performed on a representative CD4⁺ T cell clone stimulated with immobilized anti-CD3 antibody in the absence (upper lane) or in the presence (lower lane) of 10⁻⁶ M RLX for 12 h. FIG. 4B shows the image analysis of the competitive PCR as described herein after.

Southern Blot Analysis

Southern blot analysis for IFN-? was carried out with a “nested” probe designed to recognize intervening sequence between primers. This probe was obtained by PCR amplification. Primers were selected using Oligo Primer Analysis Software Version 5.0 (National Biosciences, Inc. Plymouth, Minn., USA): upper primer (227) CAGGTCATTCAGATGTAGCGGATA, lower primer (512) TCATGTATTGCTTTGCGTTGGAC (Genset, Paris, France). The DNA fragment of 286 bp amplified by PCR was subcloned using pGEM-T Vector system (Promega Co. Madison, Wis., USA) according to the manifacturer's instructions, and sequenced. Sequencing of the subcloned product was performed using Sequenase version 2.0 DNA sequencing kit (USB, Cleveland, Ohio, USA). Southern blot was performed as disclosed by E. M. Southern, <<Detection of specific sequence among DNA fragments by gel electrophoresis>> in J. Mol. Biol. 1975, 98: 503-.517.

The probe was labeled with a [³²P]deoxycytidine triphosphate using Megaprime DNA Labelling System (Amersham).

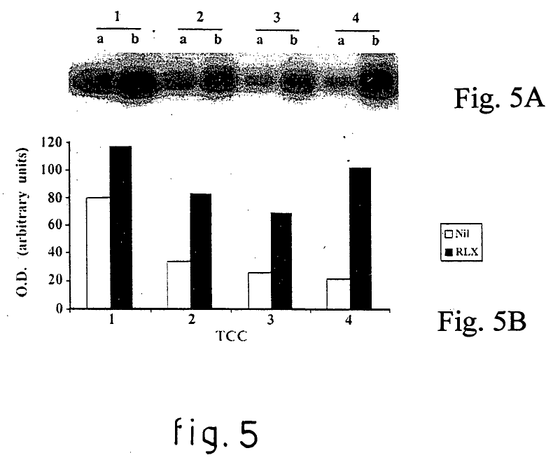

FIGS. 5A and 5B show the result of Southern blot analysis. In FIG. 5A Southern blot analysis for IFN-? was performed on four CD4⁺ T clones (labelled nos. 1, 2, 3 and 4) stimulated with immobilized anti-CD3 antibody in the absence (a) and in the presence (b) of 10⁻⁶ M RLX respectively, for 12 h. FIG. 5B shows the image analysis of the four clones performed as disclosed herein after.

Image Analysis

The intensity of the bands for ?-actin obtained by RT-PCR was measured in all the samples to confirm that the same quantities of total RNA were reverse transcribed. In addition, the intensity of the bands for IFN-? obtained by PCR amplification (MIMIC and samples) and by Southern blot was also measured. A CCD video camera C3077/01 (Hamamatsu Photonics, Tokyo, Japan) interfaced with an Apple Macintosh personal computer was used. Acquisition of images was obtained with Imagequest IQBase software (Hamamatsu Photonics). Image processing and analysis was performed with the free-share software IMAGE (National Institutes of Health, Research Services, Branch NIMH, version 1.28).

Results:

Effects of RLX on the Cytokine Profile of Short-Term Tetanus Toxoid-Specific T Cell Lines

From what has been described above, the effect of RLX on the cytokine profile of short-term tetanus toxoid specific T cell lines has been demonstrated: the production of IFN-?was significantly higher in TT-specific T cells expanded in the presence of RLX at the concentrations of 10⁻⁹, 10-8 and 10⁻⁷ M than in those generated in the absence of RLX. By contrast, no significant differences in the production of IL-4, as well as of IL-5 and IL-6, were observed between TT-specific T cells expanded in the presence or in the absence of RLX. As expected, the addition in bulk cultures of IL-12 not only resulted in a significant enrichment for TT-specific T cells able to produce IFN-?, but also significantly reduced the development of TT-specific IL-4-producing T cells.

The promoting effect of RLX on the development of IFN-?-producing TT-specific T cells was not due to a RLX-induced release of IL-12 and/or IFN-? by monocytes and/or APC present in PBMC suspensions.

Indeed, the concentrations of IL-12 and IFN-?, measured in the supernatants of adherent cell-enriched fractions of PBMC stimulated for 5 days with TT, showed no significant differences when cells were cultured in the absence or the presence of RLX. This suggests that the promoting effect of RLX on the production of IFN-? was due to a direct effect of the hormone on T cells.

Effects of RLX on the Cytokine Profile of Established CD4⁺ T Cell Clones

To provide unequivocal evidence of the direct effect of RLX on T cells, the activity of RLX on the IFN-? mRNA expression and IFN-? production by established T cell clones was examined. To this end, 12 CD4⁺ T cells clones showing a Th0-like profile (production of both IFN-? and IL-4) were cultured in the absence of any other cell type and stimulated with insolubilized anti-CD3 mAb in the presence or absence of RLX. The levels of IFN-? were significantly increased in the presence of RLX at concentrations of 10⁻⁸ M (8.20±2.27 ng/ml, p<0.03) and 10⁻⁷ M RLX (5.87±1.63 ng/m, p<0.03) in comparison with those found in the absence of RLX (3.6±0.98 ng/ml). The levels of IL-4 produced by the same CD4+T cell clones were not altered by the presence of RLX. The cytokine production by two representative T cell clones is shown in FIG. 2A.

In the same two clones, stimulated in the absence or the presence of RLX (10⁻⁸ M, the most efficient concentration assayed), IFN-? and IL-4 mRNA expression was also examined by reverse transcription (RT)-PCR analysis. Since the same amount of RNA was reverse transcribed in all the samples (as shown by the same intensity of the bands for ?-actin demonstrated by image analysis), the two clones expressed higher levels of IFN-? mRNA when RLX was added to the culture medium than in the absence of RLX. By contrast, the levels of IL-4 mRNA were the same either in the presence or in absence of RLX.

A similar effect was obtained by Southern blot analysis of IFN-? mRNA expression by four additional T cell clones stimulated with insolubilized anti-CD3 mAb in the presence or the absence of 10⁻⁸ M RLX. The RLX-induced increase of IFN-? mRNA was also quantitated by a competitive PCR technique. FIG. 4a shows the results of competitive PCR for IFN-? in a representative T cell clone (no. 2, as shown in the Southern blot), cultured in the presence or the absence of 10⁻⁸ M RLX. The image analysis revealed an approximately tenfold increase of IFN-? mRNA (from 4.1×10⁻³ to 5×10⁻² attomol/? I in the absence and in the presence of RLX, respectively).

The first experimental evidence has thus been provided, that RLX favors the in vitro development of human antigen-specific CD4⁺ T cells into Th11-like cells, i.e. cells capable of producing IFN-? but not IL-4. The promoting effect of RLX on the development of IFN-?-producing cells was not due to a RLX-induced release of IL-12 and/or IFN-? by APC. Moreover, RLX significantly increased both the expression of IFN-? mRNA and the production of IFN-? by established human T cell clones stimulated via theirTCR. Contrary to IL-12, a well-known Th1 inducer, which increases the production of IFN-? and decreases the production of IL-4 by T cells, RLX exhibits the unique property to enhance IFN-? without significantly affecting IL-4 production.

Since RLX is a hormone mainly produced during pregnancy, the first physiological implication in vivo of these findings in vitro may concern the role of RLX in the context of reactions which allow fetal tolerance by the mother's immune system. The fact that the embryo is not rejected despite fetal antigens are allogeneic to the mother is commonly referred to as the immunological paradox of pregnancy. The mechanisms that prevent rejection of fetal allotransplant are multiple and largely unknown. They include the masking of paternal histocompatibility antigens present on fetal cells, as well as the production of high concentrations of immunosuppressive substances, such as ?-fetoprotein and glycodelin. In addition, a switch from the Th1 to the Th2 profile of cytokine production at the feto-maternal interface has been suggested to play an important role in fetal protection. The enhanced production of Th2-type cytokines, such as IL-4 and IL-10, may indeed inhibit both Th1-type response and macrophage activation, thus preventing the rejection of the fetal allograft and allowing successful pregnancy.

Among the hormones of pregnancy, progesterone has been shown to promote the development of naive T cells into Th2-like cells. The data presented here, showing that RLX rather favors the development of IFN-?-producing T cells and increases the IFN-? production by established T cell clones, suggest that RLX may counterbalance the Th2-inducing activity of progesterone and promote an adequate Th1 response when the latter is required to protect the mother against dangerous intracellular pathogens or to remove aberrant placental and embryonic cells. Based on these grounds, one might ask whether RLX, due to its ability to promote a Th1-oriented immune response, may be involved in the pathogenesis of spontaneous abortion during the first trimester of pregnancy. Attempts have been made to find a correlation between RLX plasma levels and abortion risk in pregnant women. In these studies, women with failing pregnancies have been shown to have higher, equal or even lower levels of RLX compared to women who have normal pregnancies. However, it could be hypothesized that at least some pregnancy disorders might result from an imbalance between progesterone and RLX: if the RLX effect predominates, an impairment of Th2 skewing might occur, thus leading to premature rejection of the conceptus; if the progesterone effect prevails, pregnancy may be complicated by the onset or worsening of Th2-dominated diseases.

The demonstration that RLX has a Th 1-inducing capacity may provide the explanation of why this hormone has beneficial effects in some Th2-dominated disorders and opens the possibility of a future use of RLX or RLX-related drugs for the treatment of human Th2-dominated diseases. Atopic allergy is a Th2-driven hypersensitivity to innocuous antigens (allergens), inasmuch as cytokines produced by allergen-specific Th2 cells are responsible for the joint involvement of IgE-producing B cells, mast cells and eosinophils in the induction of allergic inflammation.

Interestingly, allergic asthma-like reaction and bronchial hyperresponsiveness elicited in sensitized guinea pigs has been found to be reduced by RLX treatment. The data reported here suggest that RLX may have a beneficial action on allergic inflammation not only by inhibiting mast cell granule release and decreasing leukocyte infiltration into lung tissues, but also by reducing the pathogenic Th2 response by the concurrent enhancement of Th1-oriented response.

Recently, RLX has been used in the treatment of progressive systemic sclerosis (PSS), a chronic inflammatory disorder characterized by the presence of Th2 cells in the skin and by high IL-4 levels in the serum and in other biologic fluids. Administration of IFN-? to PSS patients was found to be beneficial for the treatment of skin damage, as well as of some visceral lesions. More recently, in a control phase trial, the treatment of PSS patients with RLX resulted in the improvement of total skin score. This effect was attributed to the ability of RLX to promote collagenase activity, to inhibit collagen synthesis byfibroblasts and to prevent the fibrosis of skin and lung.

The above discussed results, however, suggest that RLX may be efficacious in the treatment of PSS also because of its capacity to inhibit the pathogenic Th2 response via the induction of endogenous IFN-? production, especially considering that RLX treatment has been found to be better tolerated than administration of IFN-? itself.

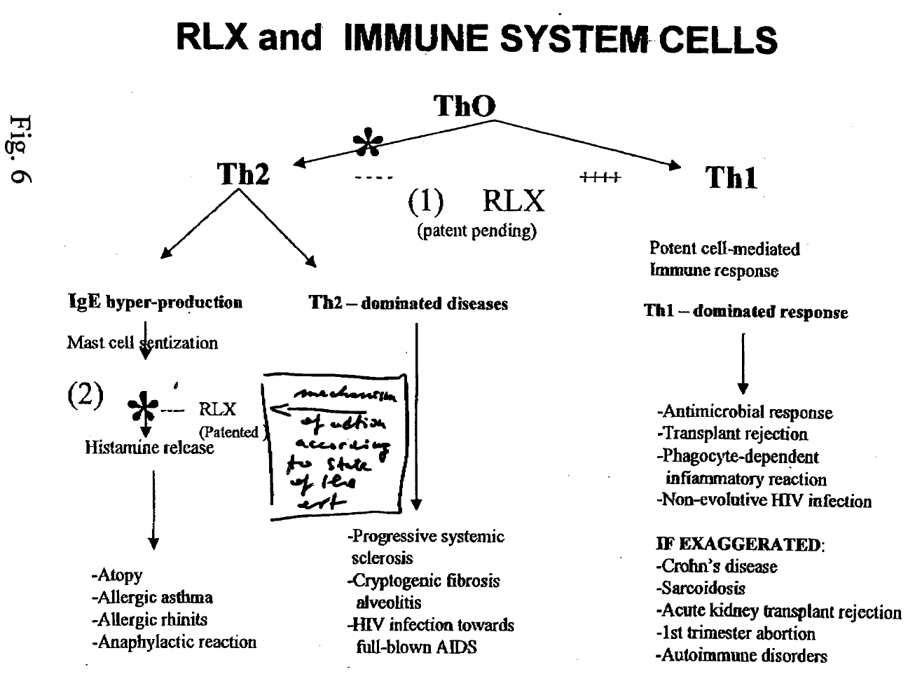

FIG. 6 shows effect of RLX. Prior knowledge has been limited to the potential effect of RLX on histamine release and thus on disorders where such phenomenon is involved. This is shown on the left of FIG. 6, the mechanism of action. On the right, the mechanism of action of the invention is shown. A potent cell mediated response occurs. The present invention for the first time discloses RLX has a different effect at a different level, namely a differentiating effect on the Th0 cells: RIX promotes development of the Th0 into Th1 rather than Th2 cells (as shown on the right side of FIG. 6). This mechanism of action was not previously known. FIG. 6 shows the different levels at which RIX acts as a therapeutic agent according to the invention.

The treatment of Th1 dominated disease is not suggested as to the action of relaxin as an agent eliciting differentiation of naive Th-cells (Th0 cells) into Th1-cells rather than Th2 cells. This previously unknown effect dramatically broadens the scope of potential use of relaxin as a therapeutic agent. Asthma is only one of the various Th2-dominated diseases and disorders. There is no suggestion of the pro-differentiating effect of relaxin on Th0 cells towards Th1-cells. This contribution goes well beyond the state of the art at the time the invention was made and opens the possibility of treating a wide range of Th2-dominated diseases previously not treated by relaxin.

1. A method of treating human Th2-dominated diseases comprising administering an amount of of relaxin or a derivative thereof that is effective to promote development of the Th0 cells into Th1 cells rather than Th2 cells.

2. The method for enhencing Th-1 response of the immunological system by development of Th0 cells into Th1 cells rather than into Th2 cells further comprising inhibiting patogenic Th2 response in a human patient via the induction of endogenous IFN-γ production.

3. The method of claim 1, wherein the development of activated human T cells into Th1-like effectors is stimulated.

4. A method of treating a Th2-dominated disease in a human patient exhibiting said disease, comprising administering to said patient an effective amount of relaxin or a derivative thereof for enhencing Th-1 resporise of the immunological system to develop Th0 cells into Th1 cells rather than into Th2 cells.

5. The method of claim 4 comprising administering to the patient an effective amount of relaxine for inducing endogenous IFN-γ production for enhencing Th-1 response of the immunological system by development of Th0 cells into Th1 cells rather than into Th2 cells

6. A method of treating a Th2-dominated disease in a human patient exhibiting said disease, the method comprising:
providing a pharmaceutical composition for stimulating the development of activated human T cells into Th1-like effectors; and
administering to said patient an effective amount of the pharmaceutical composition to provide development of Th0 cells into Th1 cells rather than into Th2 cells.
